(19) United States
(12) Patent Application Publication
(10) Pub. No.: US 2016/0345809 A1
(43) Pub. Date: Dec. 1, 2016

(54) APPARATUS, SYSTEMS AND METHODS WHICH CONTROLS AND FACILITATES INFORMATION GATHERING USING A TETHERED CAPSULE CATHETER

(71) Applicant: The General Hospital Corporation, Boston, MA (US)

(72) Inventors: Guillermo J. Tearney, Cambridge, MA (US); Michalina Gora, Cambridge, MA (US); Robert Carruth, Arlington, MA (US)

(73) Assignee: The General Hospital Corporation, Boston, MA (US)

(21) Appl. No.: 15/113,982

(22) PCT Filed: Jan. 28, 2015
(86) PCT No.: PCT/US15/13326

§ 371 Date: Jul. 25, 2016

Related U.S. Application Data
(60) Provisional application No. 61/932,446, filed on Jan. 28, 2014.

(51) Int. Cl. A61B 1/04; A61B 1/045; A61B 5/07; A61B 5/06; A61B 1/00; A61B 5/00

(52) U.S. Cl. CPC A61B 1/041; A61B 1/00147; A61B 5/0066; A61B 5/0068; A61B 5/0075; A61B 5/073; A61B 5/42; A61B 5/065; A61B 1/00009; A61B 1/045; A61B 1/00117

(57) ABSTRACT

Exemplary apparatus can be provided which includes a capsule catheter first arrangement, a tether second arrangement which connected to the first arrangement; and a weighted structure connected to or provided in the first arrangement. For example, the weighted structure can have a particular weight that causes the first arrangement to interact with gravity. In this exemplary manner, that the first arrangement can be controllably translated and positioned within a luminal anatomical structure and/or provide information about anatomic features of imaged organ.

### CROSS REFERENCE TO RELATED APPLICATION(S)

The present application relates to and claims priority from U.S. Provisional Patent Application Ser. No. 61/932,446 filed Jan. 28, 2014, the disclosure of which is incorporated herein by reference in its entirety.

### FIELD OF THE DISCLOSURE

The present disclosure relates to catheters, and more specifically to exemplary embodiments of apparatus, systems and methods which controls and/or facilitate gravity controlled administration and/or transit of a tethered capsule catheter.

### BACKGROUND INFORMATION

Catheters have been utilized in the past to obtain information regarding tissues within and/or from the surface of a body or organ system. A number of catheters have been provided, including capsule catheters, including those which are tethered. However, controlled motion of such capsule catheters may not have been easily effectuated.

Accordingly, there may be a need to address and/or overcome at least some of the above-described issues and/or deficiencies.

### SUMMARY OF EXEMPLARY EMBODIMENT

To that end, exemplary embodiments of apparatus, systems and methods can be provided which can facilitate gravity controlled administration and transit of a tethered capsule catheter.

According to an exemplary embodiment of the present disclosure, apparatus, device and method can be provided which can assist and facilitate imaging of biological tissues, e.g., luminal organs in vivo, using optical techniques. The exemplary apparatus, device and method can utilize a tethered capsule catheter that is administered and positioned using gravity, which can also provide a way to determine the anatomic location of the capsule in the complex luminal organs.

In one exemplary embodiment of the present disclosure, the tethered capsule catheter can comprise a swallowable capsule connected to the distal end of a long, flexible, small diameter tether. The tether can facilitate a transmission, including, e.g., an electrical connectivity and/or a mechanical transmission, of the electro-magnetic radiation (e.g., light) to the tissue and back to the imaging system using, e.g., an enclosed optical fiber. The tether can also be used for navigating, translating, recording and/or controlling the capsule location in the esophagus, and/or removing of the catheter. According to one exemplary embodiment of the present disclosure, to make the catheter swallowable in a comfortable manner for a patient, the capsule diameter can be below about 14 mm and a ratio between the diameter of the capsule and the diameter of a flexible tether should be over about 4:1, respectively. With a reduced size of the capsule diameter and/or an increased flexibility of the tether, e.g., the capsule weight can be increased to support the natural propulsion of peristalsis with gravity effect for an improved control of the capsule's transit and/or positioning.

According to an exemplary embodiment of the present disclosure, an exemplary apparatus can be provided which includes a capsule catheter first arrangement, a tether second arrangement which connected to the first arrangement; and a weighted structure connected to or provided in the first arrangement. For example, the weighted structure can have a particular weight that causes the first arrangement to interact with gravity. In this exemplary manner, that the first arrangement can be controllably translated and positioned within a luminal anatomical structure.

In addition, a computer can be provided which is configured or specifically programmed or modified to receive data associated with the electromagnetic radiation(s), and generate at least one image of the portion(s) of the luminal anatomical structure using the data. The computer can be configured, programmed and/or specifically modified to receive data from the capsule and/or the tether, and determine a velocity of the capsule within the luminal anatomical structure based on the data. A further arrangement can also be provided that is configured to determine a velocity of the capsule within the luminal anatomical structure. In addition, a position control arrangement can be provided which is configured to facilitate an adjustable control of a position of the first arrangement using the velocity. The computer can be configured, programmed and/or modified to determine an orientation of at least one segment of the luminal anatomical structure using the velocity.

According to another exemplary embodiment of the present disclosure, a determination and/or understanding of how gravity influences the transit of the capsule arrangement can be obtained. For example, it is possible to differentiate between different segments of imaged organ. Depending if the capsule arrangement is situated in a horizontal or vertical part of the organ, the exemplary capsule arrangement can travel (and/or caused to travel) with different velocities. From such exemplary measurements and/or determinations, it is possible to trace back and/or determine an anatomic shape of the organ, possibly image such organ, and/or facilitate an organ anatomic features characterization.

These and other objects, features and advantages of, the exemplary embodiments of the present disclosure will become apparent upon reading the following detailed description of the exemplary embodiments of the present disclosure, when taken in conjunction with the appended claims.

### BRIEF DESCRIPTION OF DRAWINGS

Further objects, features and advantages of the present disclosure will become apparent from the following detailed description taken in conjunction with the accompanying drawings showing illustrative embodiment of the present disclosure, in which:

FIG. 1A is a schematic representation of forces acting on a capsule according to an exemplary embodiment of the present disclosure during swallowing;

FIG. 1B is a schematic representation of forces acting on the exemplary capsule of FIG. 1A during traversing in the gastrointestinal (GI) tract;

FIG. 2 is a side view of a diagram of an imaging system according to an exemplary embodiment of the present disclosure which can include a tethered capsule catheter with an additional weight;

FIG. 3 is a table illustrating a summary of exemplary results from human pilot clinical trial using four exemplary prototypes of the tethered capsule catheter showing influence of the capsule outer diameter and weight on procedure safety and efficiency, according to an exemplary embodiment of the present disclosure; and

FIG. 4 is a set of illustration of exemplary results from a human pilot clinical trial using tethered capsule catheter using a gravity controlled transit that was used for an exemplary characterization of the anatomic features of duodenum.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures and provided in appended claims.

### DETAILED DESCRIPTION OF EXEMPLARY EMBODIMENTS

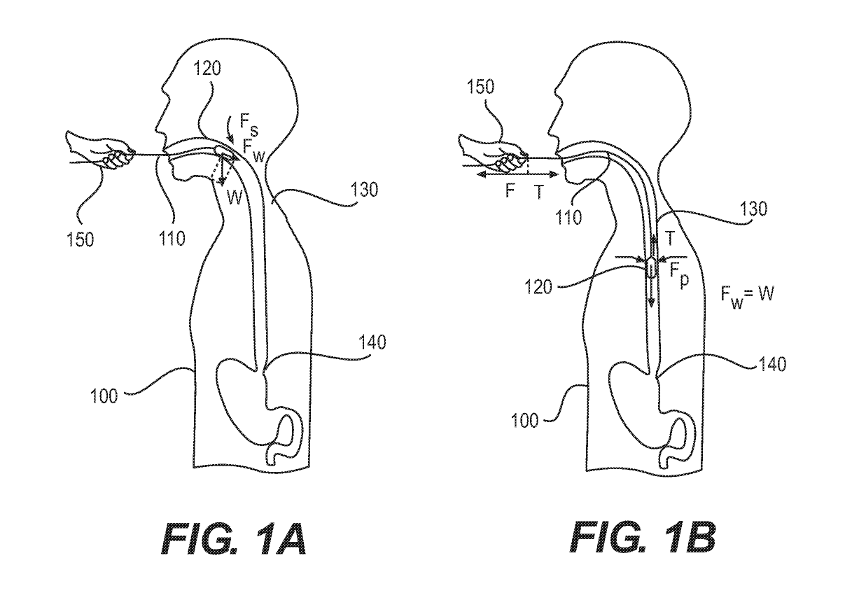

FIG. 1A-1B show schematic representations of main forces acting on a capsule 120 according to an exemplary embodiment of the present disclosure, e.g., during an exemplary imaging procedure of the upper gastrointestinal tract. During swallowing of a capsule 120, as shown in FIG. 1A, two forces related to swallowing and gravity, F_{S}, W, respectively, can influence and/or effect the movement of the capsule 120 towards the throat. In case when a tether 110 is flexible, e.g., during the swallowing, the operator 150 is not applying any force on the tether 110. Tension T on the tether 110 from the capsule 120 can be very small at that point. The force of gravity is acting straight downward on the capsule 120, and only component of the gravitational force along the slope (e.g., approximately parallel to the motion direction of the capsule 120) can pull the capsule 120 in. This force can increase with the slope angle and the weight of the capsule 120. To improve comfort and efficiency of swallowing the capsule 120, the weight of the capsule 120 can be increased and the swallowing force F_{S} can be used to support only the process of the transit and/or translation of the capsule 120. If the tether 110 is stiff enough to transfer push forces to the capsule 120, the operator 150 can use an external force on the tether 110 to facilitate faster swallowing.

FIG. 1B illustrates schematic representation of main forces acting on the exemplary capsule 120 in luminal organs of GI tract, e.g. in the esophagus 130. The transit and/or translation of the capsule 120 can mostly be dependent on a combination of natural peristalsis force F_{P} of the esophagus 130 pushing the capsule 120 down the GI track, the component of the gravity force F_{W} at least approximately and likely substantially parallel to the direction of the motion of the capsule 120 and the outside tension F applied by the operator 150 on the capsule 120 by pulling on the tether 110.

The capsule 120 can add tension T to the tether 110 Which can then act with the tension on the operator 150. The peristalsis force F_{P} acting on the walls of the capsule 120 can add friction between the capsule 120 and the tissue. This force can be proportional to the contact surface coefficient of friction multiplied by the normal force perpendicular to the capsule motion direction. This normal force can comprise vector components of the capsule 120 weight and annular compression forces of the esophageal wall. Without F, the capsule 120 can move down the GI tract and its acceleration can mostly be dependent on the capsule 120 weight and peristalsis of the subject.

Taking into account that in many cases, the weight of the capsule 120 can be constant, and if the gravity force proportional to the weight is smaller than peristalsis force, the acceleration of the capsule 120 can be used for measurement of the peristalsis force along the GI tract. In the case when the controlled capsule transit is preferred and/or required, the weight of the capsule 120 can be increased to overcome peristalsis force, and than it can be balanced by the operator 150 by applying outside force F_{OP} on the tether 110. In this case, peristalsis can be used for providing good contact with the capsule wall if necessary or preferable for quality of images.

At a distal end of the esophagus 130, the exemplary capsule 120 can reach a lower esophageal sphincter (LES) 140. Once in the LES 140, the strong sphincter muscle force can rapidly push the capsule 120 into the stomach. However, the capsule 120 first has to reach the center of the narrowed lumen of the LES 140. If the weight of the capsule 120 is not sufficient, this exemplary process can again depend on subject's peristalsis. Similarly to the swallowing and esophageal transit/translation, the force F_{W} related to the gravity can be important to facilitate an entry of the capsule 120 into the stomach, e.g., through a lower esophageal sphincter or any other sphincters, e.g. pylorus.

Through the gravity force F_{W} and/or friction, the acceleration of the capsule 120 can depend on the direction of motion of the capsule 120. If the operator 150 of the capsule 120 is not applying any outside force on the tether 110 and the weight of the capsule 120 is greater than peristaltic forces, then information about the acceleration of the capsule 120 can be used to determine orientation of the luminal organ in which the capsule 120 is moving. Such exemplary information can be used for correct reconstruction of acquired images according to anatomy of the organ.

During a pull-back of the capsule 120 through GI tract the gravity force F_{W} and the peristalsis force F_{P} can act against the direction of motion of the capsule 120. Indeed, these forces can be balanced by, e.g., an additional, controlled pulling force F on the tether 110 from the catheter operator 150. Additionally peristaltic force F_{P} can act mostly in a direction that is perpendicular to the transit/translation direction of the capsule 120, even though the average pressure of 100 mmHg in the esophagus corresponding to almost 9 N can be mostly responsible for tissue compressing on the walls of the capsule 120. For example, a small fraction of that force can act in the direction of the motion.

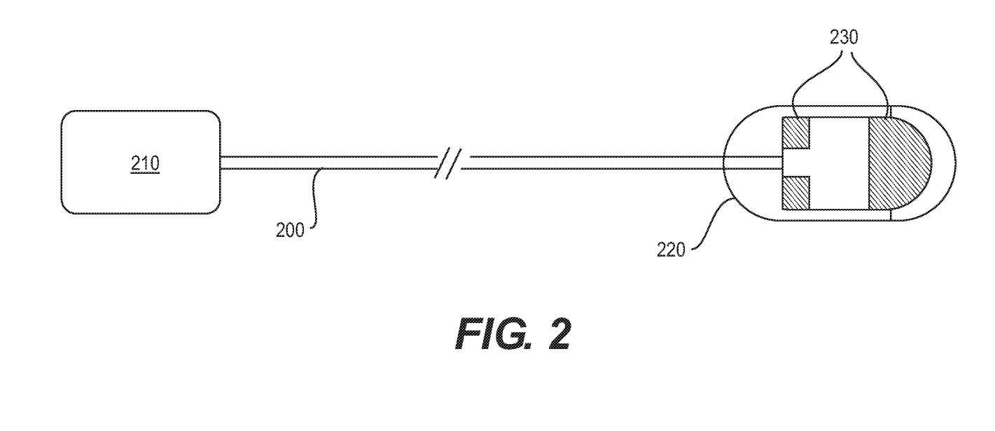

FIG. 2 shows a side view of a diagram of an optical imaging capsule catheter system/apparatus with the tether according to an exemplary embodiment of the present disclosure. This exemplary system/apparatus can include a microstructural imaging system 210 and an optical imaging catheter, which can comprise a tether 200 and a capsule 220 with an optical probe and/or an internal camera. The microstructural imaging system 210 can utilize one or more of optical frequency domain imaging, optical coherence tomography, spectral domain OCT, confocal microscopy, spectrally-encoded confocal microscopy, two photon microscope, second harmonic microscopy, third harmonic microscope, CARS, stimulated Raman microscopy, etc. For example, such imaging system 210 can be used and/or itself detect at least one electro-magnetic radiation (e.g., including but not limited to a remitted light) from any object or tissue surrounding the capsule 220 to acquire and/or determine information regarding microstructures on or in the object or tissue. If an outer diameter of the capsule 220 is decreased, additional mass 230, for example, made out or at least partially composed of high density materials like tungsten, steel, cobalt etc. can be added to the body of the capsule 220 in order to increase influence of gravity on capsule's transit. In another exemplary embodiment of the present disclosure, medical grade alloy material (e.g., medical grade metals Tungsten, Stainless Steel 316, etc) can be used to make the parts and/or components of the capsule 220 or the capsule itself, which do not interfere with imaging capabilities of the catheter.

In one exemplary embodiment of the present disclosure, the mass 230 itself and its inertial manipulation within an endoscopic probe or pill (e.g., the capsule) 220 can result in a fine tuned control of the orientation of the capsule 220, rotation and/or speed of the translation thereof through a luminal organ. By positioning the mass 230 within the capsule 220 in the distal portion and/or the proximal portion thereof, a forward pitch and/or a backward pitch of a body of the capsule 220 can be achieved. Distal mass concentrations can provide for smoother swallowing and guidance of the tip via gravity, while proximal mass concentrations can ensure straighter orientation of the imaging window to the luminal organs physiology. A translating mass 230 can be manipulated to change the balance of force from front to back to alter or tune these effects. The mass 230 can be manipulated to an angular position or velocity to induce a change in an angular position of a wall of the capsule 220. A further exemplary manipulation in succession can be used, in the driving action of the capsule 220, to induce the capsule 220 to move through or past a stricture, sphincter, obstruction and/or horizontal section of the luminal organ. An additional exemplary internal force manipulation can be applied to the mass 230 by, e.g., adding springs, motors, actuators and/or pull wires.

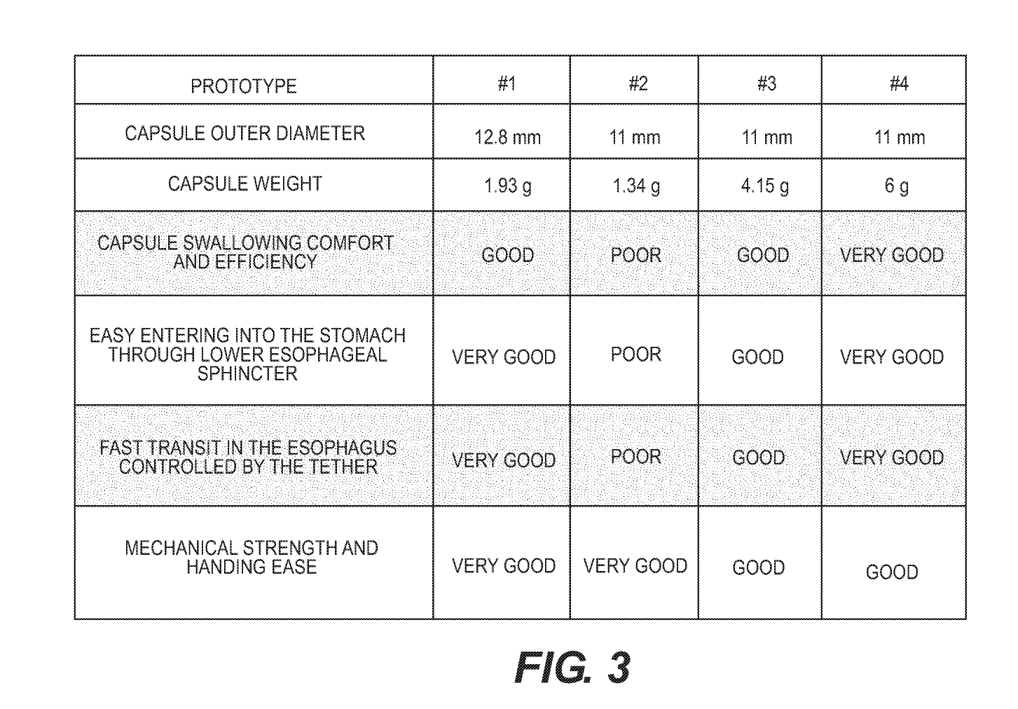

FIG. 3 illustrates a table providing exemplary results obtained in a pilot human study with the tethered capsule catheter. For example, to provide an preferable transit, translation and/or control in subjects with low or disabled peristalsis, it may be beneficial to provide an accurately adjusted capsule weight which facilitates a large enough gravity force F_{W} to balance the tether tension T can be important. Four clinical prototypes with different outer diameter and weight of the capsule were tested. Smaller outer diameter improves swallowing comfort, however it requires increasing weight to the capsule in order to increase gravity force to compensate for lower peristalsis force. That can facilitate an improved swallowing comfort, a more controlled transit in the esophagus, and an easier passing through the sphincters. An increased weight of the capsule would likely benefit from a more careful handling to prevent from extensive bending and kinking of the tether.

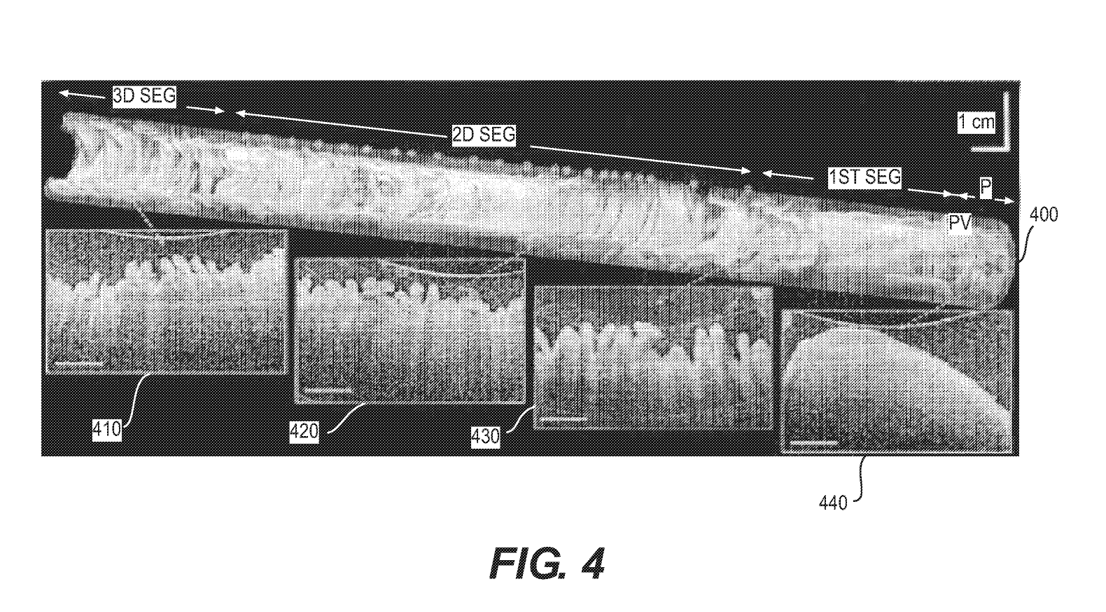

FIG. 4 illustrates exemplary results obtained in a pilot human study with the tethered capsule catheter used for imaging of first 2-cm of small intestine, called duodenum. The upper image 400 is a three-dimensional rendering of the entire pullback dataset, generated from 2500 cross-sectional images and acquired in 2 minutes. The dataset contains four anatomical sections: the pylorus (p), and 1st, 2d, and 3d duodenal segments. The pyloric valve (pv) can be clearly seen as a circular ring between the pylorus and the first segment (duodenal bulb). The first duodenal segment measures 5 cm and is notable for an absence of circular folds. The second 15-cm-long segment is comprised of multiple circular folds, the locations of which are denoted by blue asterisks. The circular folds in the third segment are much larger than those in the second segment. Superior and inferior duodenal flexures (yellow and green asterisks, respectively) appear as pockets in the duodenal surface. Dotted lines emanating from different locations in the 3D image point to corresponding exemplary cross-sectional OCT images 410, 420, 430, 440. Cross-sectional OCT images from all segments of the duodenum proper show detailed villous microscopic morphology whereas the image from the pylorus shows the typical pit and crypt architecture of the stomach. Scale bars in cross-sectional OCT images, 1 mm. Anatomical information about duodenum was obtained using gravity-controlled transit of the capsule. This exemplary method can leverage the following, e.g.: (a) the different segments of the duodenum are defined by a change in its direction, and (b) gravity affects the speed at which the capsule descends. For example when the patient is sitting or standing, if the capsule is in the vertical descending duodenum (e.g., a second segment), the capsule's velocity is high relative to when it is in the horizontal first and third segments where its velocity is much lower.

Using this principle in an exemplary pilot human study, it was possible to sense and/or determine the position of the capsule, as follows. For example, subjects were imaged while sitting upright. When the capsule was at the pylorus, the tether was reeled in to ensure that it was under slight tension and not coiled during imaging. As the capsule descended the duodenum, the operator continuously recorded the distance from the capsule to the incisors by visualizing cm marks on the tether. The displacement versus time curve was plotted; tether displacement values at velocity inflection points on this curve corresponded to transitions to new duodenal segments.

During a constant velocity TCE pullback, tether displacement marks and frame numbers were continuously recorded, allowing us to obtain a series of evenly spaced, cross-sectional OCT images where each image had a known anatomic location within the duodenum. Three-dimensional reconstructions generated using this constant velocity pullback data can then be reconstructed in the context of the subject's anatomy, confirmed by visualizing known anatomical landmarks seen in the TCE datasets (e.g. pyloric valve, ampulla, and flexures). These exemplary results indicate that anatomic positional location of the capsule can be determined using a simple tether tracking method in the gravity controlled capsule system.

The exemplary tether tracking can be performed, for example, using at least one electro-magnetic radiation reflected off of the tether and continuously detected using a detector or video camera. Velocity and displacement of the tether can be computed by cross-correlating sequential measurements. The exemplary tether tracking system according to exemplary embodiments of the present disclosure can be held by the operator or mounted to a bite block or headset that can be affixed to the subject during the exemplary procedure.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly being incorporated herein in its entirety. All publications referenced above can be incorporated herein by reference in their entireties.

What is claimed is:

1. An apparatus, comprising:
a capsule catheter first arrangement;
a tether second arrangement which connected to the first arrangement; and
a weighted structure connected to or provided in the first arrangement have a particular weight that causes the first arrangement to interact with gravity so that the first arrangement is at least one of controllably translated or controllably positioned within a luminal anatomical structure.

2. The apparatus according to paragraph 1, wherein the second arrangement includes a waveguiding arrangement which is configured to transceive at least one electromagnetic radiation between an end portion of the second arrangement and at least one portion of the luminal anatomical structure through the first arrangement.

3. The apparatus according to claim 2, further comprising a computer which is configured to receive data associated with the at least one electromagnetic radiation, and generate at least one image of the at least one portion of the luminal anatomical structure using the data.

4. The apparatus according to claim 1, further comprising a computer which is configured to receive data from at least one of the capsule or the tether, and determine a velocity of the capsule within the luminal anatomical structure based on the data.

5. The apparatus according to claim 1, further comprising a further arrangement configured to determine a velocity of the capsule within the luminal anatomical structure.

6. The apparatus according to claim 5, further comprising a position control arrangement which is configured to provide an adjustable control of a position of the first arrangement using the velocity.

7. The apparatus according to claim 5, further comprising a computer which is configured to determine an orientation of at least one segment of the luminal anatomical structure using the velocity.
